SEQUENCE LISTING (4 sequences)

SEQ ID NO: 1 - DNA (genomic), 7 bp; single-stranded, linear
  - 1..7: /note= “consensus AP-1 site (or AP-1 response element)”
tgastca

SEQ ID NO: 2 - DNA (genomic), 15 bp; single-stranded, linear
  - 1..15: /note= “consensus estrogen response element”
aggtcacagtgacct

SEQ ID NO: 3 - DNA (genomic), 7 bp; single-stranded, linear
  - 1..7: /note= “unmutated consensus AP-1 response element”
tgagtca

SEQ ID NO: 4 - DNA (genomic), 7 bp; single-stranded, linear
  - 1..7: /note= “mutated AP-1 response element from Coll517mAP-1-CAT”
gtactca

(19) United States
(12) Patent Application Publication
(10) Pub. No.: US 2002/0098477 A1
(43) Pub. Date: Jul. 25, 2002

(54) METHODS FOR SCREENING NUCLEAR TRANSCRIPTION FACTORS FOR THE ABILITY TO MODULATE AN ESTROGEN RESPONSE

(76) Inventors: PETER J. KUSHNER, SAN FRANCISCO, CA (US); ROSALIE M. UHT, SAN FRANCISCO, CA (US); PAUL WEBB, SAN FRANCISCO, CA (US)

Correspondence Address: LAW OFFICES OF JONATHAN ALAN QUINE, P O BOX 458, ALAMEDA, CA, 94501

(21) Appl. No.: 09/103,355

(22) Filed: Jun. 23, 1998

Related U.S. Application Data
(60) Provisional application No. 60/051,309, filed on Jun. 30, 1997.

(51) Int. Cl. C12Q 1/68; C12Q 1/02; C12N 1/00; G01N 33/53

(52) U.S. Cl. 435/6; 435/810; 435/975

(57) ABSTRACT

This invention provides methods of screening a nuclear transcription factor ligand for the ability to modulate estrogen activation at an AP-1 site. The methods involve the steps of: a) providing a first cell containing an estrogen receptor, a receptor for the nuclear transcription factor ligand, and a promoter comprising an AP1 site which regulates expression of a first reporter gene; b) contacting the first cell with the transcription factor ligand and with a compound having AP1 mediated estrogenic activity; and c) detecting expression of the first reporter gene

### CROSS-REFERENCE TO RELATED APPLICATIONS

This application is a continuation of provisional application U.S. Ser. No. 60/051,309, filed on Jun. 30, 1997 which is herein incorporated by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention relates to the field of signaling and induction of transcription by nuclear transcription factor ligands. In particular, this invention pertains to the modulation of estrogen activity at the AP1 site by nuclear transcription factor ligands.

### BACKGROUND OF THE INVENTION

The modulation of estrogenic activity has complex effects which may be beneficial or harmful. For example, agonistic activity may have beneficial effects, such as preventing osteoporosis and reducing serum cholesterol. (Love et al. (1992) New Eng. J. Med. 326: 852-856; Love et al. (1990) J. Natl. Cancer Inst. 82: 1327-1332). Conversely, agonist activity may also be harmful. For example, tamoxifen, an estrogen in certain contexts, sometimes increases endometrial tumor incidence (Iino et al. (1991) Cancer Treat. & Res. 53: 228-237) or switches from inhibition to stimulation of estrogen dependent growth in breast tumor progression (Parker, M. G. (ed) (1992) Cancer Surveys 14: Growth Regulation by Nuclear Hormone Receptors, Cold Spring Harbor Laboratory Press).

The modulation of estrogen mediated activation of transcription is a complex process. Estrogen action is opposed by progestins and glucocorticoids in several physiologic and pathophysiologic processes. For example, estrogen stimulates uterine growth and DNA synthesis, while glucocorticoids block these uterotrophic effects (Bibsby (1993) J. Steroid Biochem. Molec. Biol., 45: 295-301). In the stress response, estrogen treatment is associated with increased levels of circulating corticosterone (Burgess et al. (1992) Endocrinol., 131: 1261-1269), whereas glucocorticoids down-regulate hypothalamic pituitary-adrenal axis activation to reduce circulating glucocorticoid levels. Estrogen treatment is also associated with lesion-induced neuronal sprouting in vivo (Morse et al. (1992) Exptl. Neurol., 118: 47-52) and neurite outgrowth in culture (Toran-Allerand (1996) J. Steroid Biochem. Molec. Biol., 56: 169-178). Conversely, glucocorticoids in excess are associated with dendritic atrophy and cell death in pyramidal neurons of the hippocampus (Sapolsky et al. (1990) J. Neurosci., 10: 2897-2902). In bone, estrogen blocks osteoclast development and activity (Oursler et al. (1994) Proc. Natl. Acad. Sci., USA, 91: 5227-5231). Conversely, the glucocorticoid agonist dexamethasone (Dex) induces osteoclast formation (Shuto et al. (1994) Endocrinol, 134: 1121 -1126). In breast cell lines, estrogen promotes growth, while glucocorticoids inhibit it (Zhou et al. (1989) Mol. Cell. Endocrinol., 66: 189-197).

Ligands for nuclear transcription factors (particularly steroid factors), their antagonists, and analogues of such ligands are used in the treatment of a wide variety of pathological conditions. Thus, for example, among other uses, antiestrogens (e.g., tamoxifen) are used in the treatment of breast cancer (Sutherland et al. (1987) Cancer Treat. Revs., 15: 183-194), estrogens are used in the treatment of osteoporosis, progestins and estrogens are used in the regulation of fertility, and glucocorticoids are used in the treatment of certain anemias.

As explained above, nuclear transcription factor ligands vary in their ability to interact with estrogen pathway, and with their activity, depending on physiological state of the organism, tissue or cell location, and other poorly understood factors. It is thus desirable to identify or clarify the modes of activity of known nuclear transcription factor ligands, and agonistic or antagonistic analogues of such ligands. It is also desirable to identify new transcription factors ligands whose activity is better understood, and/or shows less interaction with other transcription factors, and/or is more predictable in mode of action.

The prior art fails to provide methods for quickly and easily testing nuclear transcription factor ligands or ligand analogues for the specificity of their activity and/or their interaction with estrogen activation of a target gene. This invention addresses this and other problems in the art.

### SUMMARY OF THE INVENTION

The present invention provides methods for screening nuclear transcription factor ligands (e.g, progestins, glucocorticoids, retinoic acid, vitamin D, prostaglandins, mineralcorticoids, and their analogues) for the ability to modulate estrogen activation at an AP-1 site. In one embodiment, the method includes the steps of: a) providing a first cell containing an estrogen receptor, a receptor for the nuclear transcription factor ligand, and a promoter comprising an AP1 site which regulates expression of a first reporter gene; b) contacting the first cell with the transcription factor ligand and with a compound having AP1 mediated estrogenic activity; and c) detecting expression of the first reporter gene.

In another embodiment, the method can entail detection of the transcription factor ligand's ability to activate a gene under control of an estrogen response element (ERE). In this embodiment, the method may further include the steps of: d) providing a second cell containing an estrogen receptor, a receptor for the nuclear transcription factor ligand (e.g., its cognate receptor), and a promoter comprising an estrogen response element (ERE) that regulates expression of a second reporter gene; e) contacting the second cell with the transcription factor and with the compound having AP-1 mediated estrogenic activity; and f) detecting expression of the second reporter gene. In an alternative embodiment, steps d) through e) can be performed as a completely independent assay. When performed as a component of the assay including steps a) through c), steps d) through e) may still be performed using a different cell. In this embodiment the assay would entail the use of two different cell types; one cell containing the first reporter gene construct and a second cell containing the second reporter gene construct. Alternatively, both constructs can be contained within a single cell.

In still another embodiment, the methods may also entail detection of the transcription factor ligand's ability to activate a gene under control of its own response element (e.g., in the presence of a compound having estrogenic activity). In this embodiment, the method may further include the steps of: d) providing a second cell containing a cognate receptor of the transcription factor ligand, and a promoter comprising an response element for the cognate receptor that regulates expression of a second reporter gene; e) contacting the second cell with the transcription factor ligand and with the compound having AP-1 mediated estrogenic activity; and f) detecting expression of the second reporter gene. In an alternative embodiment, steps d) through e) can be performed as a completely independent assay. When performed as a component of the assay including steps a) through c), steps d) through e) may still be performed using a different cell. In this embodiment the assay would entail the use of two different cell types; one cell containing the first reporter gene construct and a second cell containing the second reporter gene construct. Alternatively, both constructs can be contained within a single cell.

The methods may be practiced with virtually any cell. Preferred cells include one or more AP-1 proteins (e.g., jun or fos). The proteins can be endogenous to the cell, the cell can be recombinantly engineered to express the protein(s) (e.g., transfected with an expression cassette capable of expressing an AP-1 protein), or exogenous AP-1 proteins can be supplied to the cell.

Similarly, in one embodiment, the ER receptor can be endogenous to the cell, and/or the cell can be recombinantly engineered to express an ER receptor (e.g., transfected with an expression cassette that expresses an ER receptor). The ER receptor can be a native ER receptor or a modified ER receptor as described herein.

The transcription factor ligand can be virtually any nuclear transcription factor ligand as long as the cell contains a cognate receptor for that ligand. It will be appreciated that the transcription factor is a factor ligand other than the compound having AP-1 mediated estrogenic activity so that the cell is in effect contacted with two different transcription factor ligands, the transcription factor ligand and the compound having AP-1 mediated estrogenic activity. As with the ER receptor, the transcription factor (e.g., the cognate receptor of the transcription factor ligand) can be one that is endogenously expressed by the cell and/or the cell can be recombinantly engineered to express the receptor. Preferred transcription factor ligands and their cognate receptors (transcription factors) include, but are not limited to a glucocorticoid and a glucocorticoid receptor (GR), a protestin (eg., progesterone) and a progestin receptor, retinoic acid and a retinoic acid receptor, vitamin D, and a vitamin D receptor, an androgen and an androgen receptor, a mineralcorticoid and a mineralcorticoid receptor, and so forth.

Virtually any reporter gene is suitable, however in a preferred embodiment, the reporter gene is a luciferase, a green fluorescent protein (GFP) or a β-galactosidase. Preferred promoter/reporter gene combinations include, but are not limited to, any of the promoter/reporter gene combinations described herein. It will be appreciated that where the first and second reporter gene are located in different cells, both reporter genes can be the same type of reporter gene. Conversely, where both reporter genes are located in the same cell, the reporter genes are preferably different reporter genes (e.g., providing different distinguishable signals).

Suitable cells include, but are not limited to HeLa cells, MCF-7 cells MDA453 cells, ZR-75-1, ERC1 cells, ERC2 cells, or ERC3.

In another embodiment, this invention provides methods of screening an agent (e.g., a test compound that is to be screened for agonistic or antagonistic transcription factor activity (e.g., a steroid or steroid analogue, or steroid inhibitor)) for the ability to alter modulation of estrogen activation at an AP-1 site by a nuclear transcription factor. These methods are performed as described above except that the contacting step also involves contacting the cell with the agent. The cell may be contacted by the agent prior to, or simultaneous with, contact with the compound having AP-1 mediated estrogenic activity and/or prior to, or simultaneous with, contact with the transcription factor.

In still another embodiment, this invention provides kits for the practice of the methods described herein. In one embodiment the kits include a first cell containing an estrogen receptor, a receptor for a nuclear transcription factor, and a promoter comprising an AP-1 site that regulates expression of the first reporter gene. The kits also preferably include instructional materials detailing protocols for the practice of the assay methods described herein.

Definitions

As used herein an antiestrogen is a compound that substantially inhibits estrogen activity as measured in a standard assay for estrogenic activity, for example, cellular assays as described in Webb et al. Mol. Endocrinol., 6:157-167 (1993). More generally, a “transcription factor antagonist” is a compound that substantially inhibits transcription factor activity as measured in a standard assay for that transcription factor activity.

A “nuclear transcription factor” as used herein refers to members of the nuclear transcription factor superfamily. This is a family of receptors that are capable of entering the nucleus of a cell and once there, effecting the up-regulation or down-regulation of one or more genes. A “nuclear transcription factor ligand” is a compound that binds to a nuclear transcription factor. Preferred nuclear transcription factors are typically steroids, however, the group is not so limited. Nuclear transcription factor ligands include, but are not limited to estrogen, progestins, androgens, mineralcorticoids, glucocorticoids, retinoic acid, vitamin D, and prostaglandins. Transcription factor ligands also include analogues of naturally occurring factors and blocking agents (antagonists) of such factors. Transcription factors also include, as they are identified, the ligands that bind orphan receptors (those nuclear transcription factors which have been identified by sequence homology, but whose ligand is yet unidentified). It will be appreciated that when used in the context of a modulator of estrogen activity, the nuclear transcription factor ligand is typically one other than estrogen (or other than the estrogen or estrogen agonist whose activity is being modulated). Nuclear transcription factors typically mediate their activity through binding of a cognate receptor in the cell nucleus. The term cognate receptor” refers to a receptor of the type that is typically bound by the transcription ligand in question. Thus, the cognate receptor for an estrogen is an estrogen receptor, the cognate receptor for a glucocorticoid is a glucocorticoid receptor, the receptor for a progestin is a progestin receptor, and so forth. The cognate receptor includes the native (naturally occurring) form as well as modified receptors.

The phrases “modulate estrogen activation” or “modulation of estrogen activation” refer to alteration of the estrogen induced expression of a particular gene. Where the phrase additionally recites “at an AP-1 site or at an ERE” the phrase refers to alteration of the level of estrogen induced expression of one or more genes under control of the AP-1 site or ERE site respectively. The phrase “detecting expression” when used with reference to a reporter gene refers to detection of presence or absence of expression of the reporter gene or to quantification of expression level of the reporter gene. The quantification can be either an absolute measurement or a relative measurement (e.g., in comparison to another expressed gene).

The term “operably linked” refers to functional linkage between a nucleic acid expression control sequence (such as a promoter, signal sequence, or transcription factor binding site) and a second nucleic acid sequence, wherein the expression control sequence affects transcription and/or translation of the nucleic acid corresponding to the second sequence.

The term “recombinant” when used with reference to a cell indicates that the cell replicates a heterologous nucleic acid, or expresses a peptide or protein encoded by a heterologous nucleic acid. Recombinant cells can express genes that are not found within the native (non-recombinant) form of the cell. Recombinant cells can also express genes found in the native form of the cell wherein the genes are modified and re-introduced into the cell by artificial means. Recombinant expression refers to the expression of the heterologous nucleic acid by such a recombinant cell.

A “heterologous sequence” or a “heterologous nucleic acid”, as used herein, is one that originates from a foreign source (or species) or, if from the same source, is modified from its original form. Thus, a heterologous nucleic acid operably linked to a promoter is from a source different from that from which the promoter was derived, or, if from the same source, is modified from its original form. Modification of the heterologous sequence may occur, e.g., by treating the DNA with a restriction enzyme to generate a DNA fragment that is capable of being operably linked to the promoter. Techniques such as site-directed mutagenesis are also useful for modifying a heterologous sequence.

A “recombinant expression cassette” or simply an “expression cassette” is a nucleic acid construct, generated recombinantly or synthetically, with nucleic acid elements that are capable of effecting expression of a structural gene in hosts compatible with such sequences. Expression cassettes include at least promoters and optionally, transcription termination signals. Typically, the recombinant expression cassette includes a nucleic acid to be transcribed (e.g., a nucleic acid encoding a desired polypeptide), and a promoter. Additional factors necessary or helpful in effecting expression may also be used as described herein. For example, an expression cassette can also include nucleotide sequences that encode a signal sequence that directs secretion of an expressed protein from the host cell.

AP-1 mediated estrogenic/agonist activity, as used herein, refers to activation of a gene under the control of an AP-1 site (also referred to as an AP-1 response element) mediated by the interaction of a nuclear transcription factor with the AP-1 site. When used in reference to ER mediated activation of a gene controlled by the AP-1 site, the pathway is referred to as the indirect estrogen response (in contrast to the classical estrogen response which is mediated through an ERE). A general description of the AP1 site is found in Angel & Kann, Biochem. Biophys. Acta., 1072: 129-157 (1991) and Angel, et al., Cell, 49: 729-739 (1987).

A “compound having AP-1 mediated estrogenic activity” refers to a compound that, when present in a cell containing a gene under control of an AP-1 site and AP-1 proteins, activates transcription of the gene under control of the AP-1 site.

### BRIEF DESCRIPTION OF THE DRAWINGS

FIGS. 1A, 1B, and 1C illustrate that estradiol and the glucocorticoid dexamethasone modulate each others transcriptional properties at the AP-1 response element. FIG. 1A shows the structure of collagenase reporter and steroid receptor vectors used. FIG. 1B shows HeLa cells co-transfected with the Coll73-LUC reporter gene (5μg) and the human ER expression vector, pHEO (5 μg) and treated were treated with vehicle, Dex, estradiol, or Dex+Estradiol (10⁻⁷M, each steroid) for approximately 40 hrs and then assayed for luciferase activity. The data are from three experiments. Columns represent the average fold induction, defined as the steroid treatment divided by the No Steroid treatment. FIG. 1C shows that three point mutations in the AP-1 site of the collagenase promoter markedly attenuated steroid effects on transcriptional activation. HeLa cells were transfected with 5 μg of either the intact (Coll517-CAT) or mutated (Coll517mAP-1-CAT) collagenase reporter genes along with GR (1 μg) and ER (3 μg) expression vectors. Data are from two experiments. Columns represent the average. (B&C) Error bars represent the standard deviation.

FIGS. 2A, 2B, 2C, and 2D illustrate ER and GR competition at the AP-1 site. HeLa cells were transfected with the Coll73-LUC reporter gene (5 μg) and the expression vectors illustrated in FIG. 1A as follows: FIG. 2A shows GR (1 μg) and increasing amounts of ER as indicated. Columns represent an average of three treatment points from one experiment. FIG. 2B shows the average of three experiments expressed as fold induction. FIG. 2C shows the results of cells transfected with ER (10 μg) and increasing amounts of GR. Columns represent the average of three treatment points. FIG. 2D shows the average of two experiments not including the experiment shown in FIG. 2C. FIGS. 2A through 2D the error bars represent standard deviation. (RLU) Relative light units.

FIGS. 3A and 3B show that Dex inhibits both Tamoxifen stimulation and the constitutive activity of the ER deleted of the ligand binding domain (HE15). HeLa cells were transfected with the Coll73-LUC reporter gene as in FIG. 1A and 1B. Cells were transfected with GR (1 μg) and increasing amounts of HE0. They were treated with vehicle, Dex, tamoxifen (5×10⁻⁶M), or Dex+tamoxifen. In FIG. 3A the columns represent the average of three treatment points. FIG. 3B shows the results of cells transfected with increasing amounts of HE15 and treated with vehicle or Dex. As a control, one set of cells was transfected with HE0 and treated with No Steroid, Dex, estradiol and Dex+estradiol. Columns represent the average of three treatment points. In FIGS. 3A and 3B the experiments were repeated 3 times or more. The error bars represent standard deviation.

FIG. 4 shows that Dex inhibits the ER deleted of its DNA binding domain. HeLa cells were transfected with the Coll73-LUC reporter gene as in FIG. 1 and expression vectors as follows: Empty expression vector (pSG5; 5 μg), ER (HE0; 5 μg), and the ER deleted of its DNA binding domain (HE11; 3 and 5 μg). Cells were treated with steroids as in FIG. 1. Columns represent an average of three treatment points, error bars the standard deviation. The data are representative of similar experiments performed 3 times or more.

FIGS. 5A and 5B show that co-transfected c-Jun potentiates steroid effects; and co-transfected c-Fos further potentiates c-Jun effects on estradiol stimulation. HeLa cells were transfected with the Coll73-LUC reporter gene and treated with steroids as in FIG. 1. Cells were co-transfected with ER and GR expression vectors (1 μg each) and increasing amounts of c-Jun (FIG. 5A) or c-Fos (FIG. 5B). All columns and error bars represent the average of three treatment points except in (B) in which the c-Jun and c-Jun+c-Fos data represent one transfection with one treatment point each. Error bars represent standard deviation. The data are representative of similar experiments performed 3 times or more.

FIGS. 6A and 6B show that PMA and TNF-α differentially alter steroid responses at Coll73.HeLa cells were transfected with the Coll73-LUC reporter gene as in FIG. 1. They were co-transfected with ER (5 μg) and GR (1 μg) and treated with steroids as in FIG. 1 in the presence or absence of PMA (FIG. 6A) or TNF− (FIG. 6B) at the doses indicated. (FIG. 6A) Note: the scale for PMA treated cells is 10× that of cells not treated with PMA (“No PMA”). Columns represent an average of three treatment points, error bars represent standard deviation. The data are representative of similar experiments performed 3 times or less. (B) Columns represent an average of three experiments. (A&B) error bars represent standard deviation.

FIG. 7 illustrates that Dex inhibits estradiol stimulation of transcription through the AP-1 response element in a hypothalamic cell line. GT1-1 cells were transfected with ColALuc (5 μg), HE0 (5 μg), GR (1 μg), and c-Jun (3 μg). Cells were treated with steroids 4 hrs. after transfection and harvested 36 hrs. later. The data are expressed as percent estradiol stimulation. Columns represent the average of three experiments. Error bars represent the standard deviation.

FIGS. 8A and 8B show that estradiol and the progestin RU5020 modulate each others transcriptional properties at the AP-1 response element. (FIG. 8A) HeLa cells were transfected with ColALuc (5 μg), ER (1 μg), PR-A or PR-B (1 μg), and c-Jun (3 μg). Data are from four separate transfections from three experiments for PR-A and from two transfections from two experiments for PR-B. Columns represent the average fold induction. (FIG. 8B) CV-1 cells were transfected with ColALuc (5 μg), ER (HE0, 0.5 μg), PR-A (1 μg) and c-Jun (3 μg). Data is from one experiment. Columns represent the average of two treatment points. Similar experiments have been repeated 3 times or less. (FIGS. 8A and 8B) Cells were treated with steroids immediately after transfection and harvested 40 hrs later. Error bars represent standard deviation.

### DESCRIPTION OF THE PREFERRED EMBODIMENT

Nuclear transcription factor ligands are used in the treatment of a wide variety of pathological conditions. Thus, for example, estrogens are used in the treatment of osteoporosis and other aspects (e.g., vasomotor instability) of menopause, in the treatment of hypoestrogenism, and in the regulation of fertility. Glucocorticoids are used in the treatment of pure red cell anemia, acute renal failure due to acute glomerulonephritis or vasculitis, lymphocytic leukemias, lymphomas, and other conditions. Progestins or progestational agents such as medroxyprogesterone or megestrol acetate are used in the treatment of endometrial carcinoma and breast carcinoma, and are used in the regulation of fertility.

The activities of these, and other, nuclear transcription factor ligands, however, are complex and vary with physiological context. Moreover the agonistic activity of the transcription factor ligand, ligand analogue agonist, or putative blocking agent (antagonist), may be mediated through a variety of pathways. Thus, for example, it has been shown that antiestrogens can exhibit agonist estrogenic activity mediated through an AP-1 response element (see, e.g., Webb, et al (1995) Mol. Endo., 9: 443-456). In addition, the transcription factor ligands are known to interact. Thus, for example, progestins and glucocorticoids are known to oppose estrogen activity in various physiologic and pathophysiologic processes.

Consequently, it is desirable, when contemplating use of a nuclear transcription factor ligand to evaluate the activity of that ligand on estrogen activated transcription. This permits the identification of transcription factor agonists or antagonists that either act on or act independently of the estrogen response. This facilitates the design and administration of therapeutics whose activity is well characterized in the physiological context in which they are to be administered. It was a discovery of this invention that liganded nuclear transcription factors can interact with estrogen activation at the level of transcription and this activity is detectable through AP-1 mediated transcription. This invention therefore provides assays (methods of screening) nuclear transcription factor ligands, and putative or known transcription factor ligand agonists or antagonists for the ability to modulate estrogen activation at an AP-1 site.

In one embodiment, the methods involve providing a cell containing an estrogen receptor, a receptor for the nuclear transcription factor (e.g., glucocorticoid (GR) receptor), and a promoter comprising an AP-1 site operably linked to a reporter gene. The cell is then contacted with a compound having estrogenic activity (e.g., β-estradiol) and with the nuclear transcription factor ligand, transcription factor ligand agonist or antagonist, (test compound that is to be screened) and the effect on estrogen mediated activation of transcription through the AP-1 site is evaluated. The level of AP-1 mediated transcription is determined by detecting presence, absence, or level of expression of the reporter gene.

In a preferred embodiment, the level of expression of the reporter gene from the assay is compared to the level of expression from a control that either lacks the transcription factor ligand (test compound) and/or to a control that contains the test compound in a known different concentration. It will be appreciated that, where the reporter gene assay does not require lysis or other destruction of the cell, the control can be performed using the same cell before or after the test assay. However, in a preferred embodiment, the control is performed using a different cell and is most preferably performed simultaneously with the test assay.

In another embodiment, this invention provides assays (methods of screening) nuclear transcription factor ligands, transcription factor ligand analogues, and potential transcription factor antagonists for the ability to modulate estrogen activation at an estrogen response element (ERE). In this embodiment, the method is identical to the screen for activation at an AP-1 site, however in this embodiment, the cell contains a promoter comprising an estrogen response element (ERE) site operably linked to a reporter gene instead of the AP-1/reporter gene construct. The assay is conducted in exactly the same manner. Detection of the presence, absence, or quantification of the reporter gene product provides a measure of the estrogen-mediated activation of transcription through an ERE (classical pathway) and of the effect of the nuclear transcription factor ligand on such estrogen mediated activation.

It will be appreciated that these assays allow one to determine the extent to which a given nuclear transcription factor ligand (e.g., a progestin, glucocorticoid, or other steroid) maintains its ability to block or agonize estrogen activation at an AP-1 site or at an ERE. By varying the estrogen agonists in these assays, it is possible to identify those estrogens (or estrogen analogues) that can or cannot be inhibited or agonized by a nuclear transcription factor ligand at an AP-1 site, at an ERE, or at both sites.

The two assays described above can be used in conjunction with each other to assay activation through both the “indirect pathway (at an AP-1 site) and the direct or classical pathway (at an ERE). Where the same reporter gene is used, such a combined assay preferably utilizes two different cell types; one cell type containing the AP-1/reporter gene construct and another cell type containing the ERE/reporter gene construct. However, different reporter genes can also be used. In this case, both assay systems may be performed in one cell. Thus, in this embodiment, a single cell type can be used containing both an AP-1/reporter gene construct (e.g., Coll73-LUC or Coll60-LUC) and an ERE/reporter gene construct (e.g., EREcoll60CAT and EREcoll73CAT, see, Webb, et al (1995) Mol. Endo., 9: 443-456, and U.S. Ser. No. 08/410,807).

In still another embodiment, this invention provides methods of screening for agents that inhibit the modulation of estrogen activation at an AP-1 or ERE site by a nuclear transcription factor ligand. The assay is performed as described above, with the addition of the agent that is to be screened. The effect of the agent on transcription factor ligand modulation of estrogen activation at the AP-1 or ERE is then determined by detecting the presence, absence, or level of expression of the reporter regulated by the AP-1 site, the reporter gene regulated by the ERE, or both. Appropriate controls in this case may include the same assay with all components except the agent being screened and/or the same assay with all components including the agent being screened at a known concentration different from that in the test assay. Again, the assay(s) may be performed with separate cells containing the AP-1/reporter gene and ERE/reporter gene constructs, or with a single cell containing both constructs.

In still another embodiment, this invention provides assays for screening an orphan receptor for the ability to modulate estrogen activation at an AP-1 site. This assay involves: providing a first cell containing an estrogen receptor, and orphan receptor, and a promoter comprising an AP-1 site that regulates expression of a first reporter gene; b) contacting the first cell with a compound having AP-1 mediated estrogenic activity; and detecting expression of the first reporter gene. The effect of the compound on orphan receptor mediated modulation at the AP-1 site is then determined by detecting the presence, absence, or level of expression of the reporter regulated by the AP-1 site. Alternatively, instead of the orphan receptor, it is possible to use a transcription activator or co-activator.

Test Cells

In animals and in man the balance between stimulatory and inhibitory activities of various nuclear transcription factors on the estrogen response varies widely depending on the organ, cell or specific protein measured as an indicator of estrogenic activity. In order for the nuclear transcription factor in question to modulate the estrogen activation at the level of transcription, a given cell would preferably contain receptors for both estrogen and the transcription factor (e.g., both ER and GR where the transcription factor in question is a glucocorticoid).

Cells that naturally express both receptor types can be used in the assays of this invention. Alternatively, cells can be modified (e.g., through recombinant DNA techniques) to express the ER and transcription factor receptor of choice.

Suitable cells for practicing the methods of this invention include, but are not limited to cells derived from a uterine cervical adenocarcinoma (HeLa), a hypothalamic cell line (GT1-1 (Mellon et al. (1990) Neuron, 5: 1-10), MCF-7 cells (ATCC No. HTB 22), MDA453 cells (ATCC No. HTB 131), ZR-75-1 cells (ATCC No. CRL 1500) or ERC1 cells described in Kushner et al., Mol. Endocrinol., 4:1465-1473 (1990). ERC2 and ERC3 cells as described by Webb, et al. Mol. Endocrinol., 6:157-167 (1993). It will be appreciated that the invention is not limited to practice in mammalian cells and may be practiced, for example in yeast and insect cells, transfected with the appropriate genes and recombinant constructs.

Cells Naturally Expressing Two or More Receptor Types.

Many cells that express a second transcription factor receptor in addition to the estrogen receptor (ER) are well known to those of skill in the art. Thus, for example, in the uterus there is evidence that ER and glucocorticoid receptors (GR) co-exist in the endometrium (Prodi et al. (1979) Tumori. 65: 241-253). In the brain, maps of ER and GR immunoreactivity and mRNA localization suggest co-localization in certain cerebral nuclei such as the paraventricular nucleus of the hypothalamus, the hypothalamic arcuate nucleus, and the central nucleus of the amygdala (Fuxe et al. (1985) Endocrinol., 118: 1803-1812; Simerly et al. (1990) J. Comp. Neurol. 294: 76-95). In bone, ER and have been found in cultured osteoblast-like cells (Liesegang et al. (1994) J. Andrology, 14: 194-199). ER has also been demonstrated in osteoclasts (Oursler et al. (1994) Proc. Natl. Acad. Sci., USA, 91: 5227-5231) and data suggest that the glucocorticoid dexamethasone (Dex) regulates metabolism in these cells (Wong (1979) J. Biol. Chem., 254: 6337-6340) raising the possibility that osteoclasts contain functional GR as well. In addition, numerous tumor cell lines have been demonstrated to have both ER and GR (Ewing et al. (1989) Int. J Cancer., 44: 744-752.

Cells Recombinantly Modified to Express Two or More Receptor Types.

Cells normally lacking the ER or the other transcription factor cognate receptor, or both, can be recombinantly modified to express both receptors and optionally, additional receptors. Typically this involves transfecting the cell with an expression cassette comprising a nucleic acid encoding the receptor and culturing the cell under conditions where the receptor is expressed (e.g., in the presence of an appropriate inducer if the promoter regulating expression of the receptor is inducible). Typically, the cassette is selected to provide constitutive expression of the receptor.

A cell that naturally expresses one receptor need only be modified to express the second receptor. However, if the cell expresses neither receptor, it may be transfected with expression cassettes expressing both receptors. Even where a cell naturally expresses one or both receptors, it may be recombinantly modified to express those receptors at a higher level (e.g., by introducing expression cassettes encoding the receptor(s) whose expression level it is desired to increase).

The cells need not contain “native” receptors, but may be modified to provide truncated or chimeric receptors to provide increased affinity and/or sensitivity of the assay. Thus, for example, Berry, et al. (1990), EMBO J., 9: 2811-2818, describe the production of cells containing truncated or chimeric ER receptors.

Methods of modifying cells to express particular receptors are well known to those of skill in the art. Thus, for example, cells modified to express high levels of estrogen receptor are described by Kushner et al. (1990), Mol. Endocrinol., 4:1465-1473. See also Hirst et al (1990) Mol. Endocrinol., 4: 162-170). Transfection of cells to express estrogen receptors (ER), glucocorticoid receptors (GR) and progestin receptors (PR) is described in Example 1.

Cells Recombinantly Modified to Express AP-1 Proteins.

The cells of this invention preferably express one or more AP-1 proteins (the Jun or Fos proteins or other members of that protein family, see bohmaan, et al. (1987) Science, 238: 1386-1392).

The cells can naturally express the AP-1 protein(s) or they can be modified (e.g., by transfection with a suitable expression cassette) to express a heterologous AP-1 protein. Methods of expressing AP-1 proteins are well known to those of skill in the art (see, e.g., Turner et al. (1989) Science 243:1689-1694 and Cohen et al. (1989) Genes & Dev., 3: 173-184, and Example 1). Cells that naturally express one or more AP-1 proteins may still be so modified to increase intracellular jun and/or fos levels.

Nuclear Receptors

As explained above, in one embodiment, the assays of this invention utilize cells containing an estrogen receptor and a receptor for a nuclear transcription factor (typically a transcription factor other than estrogen). The factor can be one that is expressed endogenously by the cell or, alternatively, the cell can be modified (e.g., a recombinant cell) so that it expresses the receptor.

Estrogen Receptor

An estrogen receptor, as used herein, includes an estrogen receptor in its native (naturally occurring) form as well as modified estrogen receptors. Numerous modifications of estrogen receptors are known to those of skill in the art. These include, but are not limited to VP16-ER, V-ER, a chimeric receptor comprising the strong VP16 transcriptional activation domain linked to the amino terminus of the ER, V-ER in which the ER DNA binding domain (DBD) is deleted, H11 an ER lacking the DNA binding domain, and the like (see e.g., Kumar et al., Cell, 51: 941-951 (1987) and Elliston et al. (1990) J Biol Chem 265:11517-21).

Nuclear Transcription Factor Ligand and Cognate Receptor

In addition to the estrogen receptor, the cells preferably contain a cognate receptor for the nuclear transcription factor ligand whose interaction with the estrogen activation pathways is to be assayed. As used herein, the term “cognate receptor” refers to a receptor of the type that is typically bound by the transcription factor ligand in question. Thus, the cognate receptor for an estrogen is an estrogen receptor, the cognate receptor for a glucocorticoid is a glucocorticoid receptor, the receptor for a progestin is a progestin receptor, and so forth. As with the estrogen receptor, the cognate receptor includes the native (naturally occurring) form as well as modified receptors.

Natural and modified cognate receptors for nuclear transcription factor ligands, particularly for steroid nuclear transcription factors, are well known to those of skill in the art. These include, but are not limited to the glucocorticoid receptors, the progestin receptors (e.g., PR-A, PR-B (see, e.g., Law et al. (1987) Proc. Natl. Acad. Sci. USA 84: 2877-2881; Wei et al. (1988) Mol. Endo. 2: 62-72; and Kushner et al. (1990) Mol. Endocrinol, 4:1465-1473), vitamin D receptors, mineralcorticoid receptors, androgen receptors, and thyroid hormone receptors (see, Mangelsdorf (1995) Cell, 83: 835-839).

Promoter/Reporter Construct

The cells of this invention are transfected with reporter genes in which a response element (either the AP-1 site or ERE) regulates expression of a reporter gene. In a preferred embodiment, two different reporter genes are used. One gene reports transcription induced by the classical estrogen response system, while the other gene reports transcription induced by the indirect estrogen response. The two reporter genes and response elements are typically placed in separate cells, but the methods can also be used with both constructs in the same cell.

Promoter Constructs.

AP-1/Reporter Construct.

In one embodiment the methods of this invention involve providing a cell containing an estrogen receptor a receptor for a nuclear transcription factor, and a promoter comprising an AP-1 site that regulates expression of a reporter gene (also referred to herein as the reporter gene for the indirect estrogen response pathway (see, e.g., U.S. Ser. No. 08/410,807 and Webb, et al (1995) Mol. Endo., 9: 443-456).

The reporter gene for the indirect estrogen response pathway contains an AP-1 site preferably upstream of the target promoter and capable of regulating (i.e., operably linked to) that promoter. The AP-1 site are sites that are bound by AP-1 (the Jun and Fos proteins) or other members of that protein family. In a preferred embodiment, the consensus AP-1 site (or AP-1 response element) is TGA(C/G)TCA (SEQ ID NO: 1).

One of skill would recognize that the particular AP1 site used is not a critical aspect of the invention. Any sequence capable of being bound by AP1 or members of that family and regulating a promoter is suitable. This would include promoters which encompass a naturally occurring AP1 site. Typical promoters include, but are not restricted to metalloprotease genes such as stromelysin, gelatinase, matrilysin, and the human collagenase gene.

Alternatively promoters may be constructed which contain a non-naturally occurring AP-1, or related, binding site. This facilitates the creation of reporter gene systems that are not typically found under the control of AP-1. In addition, promoters may be constructed which contain multiple copies of the AP-1 site thereby increasing the sensitivity or possibly modulating the response the reporter gene system.

ERE/Reporter Construct

In another embodiment, the methods of this invention involve providing a cell containing a promoter comprising an estrogen response element that regulates expression of a reporter gene (also referred to herein as the reporter gene for the direct or classical estrogen response pathway (see, e.g., U.S. Ser. No. 08/410,807 and Webb, et al (1995) Mol. Endo., 9: 443-456). This permits detection of the “direct” (classical) estrogen response and evaluation of the interaction or modulation of the classical response by the nuclear transcription factor ligand.

Typically, the estrogen response element (ERE) is upstream of the target promoter and capable of regulating that promoter. In a preferred embodiment the ERE may be the consensus estrogen response element AGGTCACAGTGACCT (SEQ ID NO: 2) from the Xenopus vitellogenin A2 gene. The particular ERE used in the cell is not a critical aspect of the invention and the present invention is not limited to the use of any one particular ERE. Suitable EREs are well known to those of skill. For instance, other sources of naturally occurring EREs include the vitellogenin B2 gene, the chicken ovalbumin gene, and the PS2 gene. Alternatively, non-naturally occurring EREs may be inserted into particular promoters. The consensus ERE from the Xenopus vitellogenin A2 gene is widely used for this purpose, but other EREs may be used as well.

Reporter Gene(s)

The present invention is not limited to a particular reporter gene. Any gene that expresses an easily assayable product will provide a suitable indicator for the present assay. Suitable reporter genes are well known to those of skill in the art. Examples of reporter genes include, but are not limited to CAT (chloramphenicol acetyl transferase) (Alton and Vapnek (1979), Nature 282: 864-869), luciferase, and other enzyme detection systems, such as beta-galactosidase; firefly luciferase (deWet et al. (1987), Mol. Cell. Biol. 7:725-737); bacterial luciferase (Engebrecht et al. (1984), Proc. Natl. Acad. Sci., USA, 1: 4154-4158; Baldwin et al. (1984), Biochemistry 23:3663-3667); alkaline phosphatase (Toh et al. (1989) Eur. J. Biochem. 182: 231-238, Hall et al. (1983) J. Mol. Appl. Gen. 2: 101), and green fluorescent protein.

One of skill will recognize that various recombinant constructs comprising the AP-1 site can be used in combination with any promoter and reporter gene compatible with the cell being used. The promoter will preferably be one susceptible to regulation by the AP-1 site.

Construction of the Promoter/Reporter Expression Cassette

The promoter/reporter expression cassettes and, other expression cassettes described herein, can be constructed according to ordinary methods well known to those of skill in the art. Construction of these cassettes is variously exemplified in Example 1, in U.S. Ser. No. 08/410,807, in Webb et al. (1995) Mol. Endo. 9: 443-456, and in other references cited herein.

The constructs can all be created using standard amplification and cloning methodologies well known to those of skill in the art. Examples of these techniques and instructions sufficient to direct persons of skill through many cloning exercises are found in Berger and Kimmel, Guide to Molecular Cloning Techniques, Methods in Enzymology 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook et al.); Current Protocols in Molecular Biology, F. M. Ausubel et al., eds., Current Protocols, a joint venture between Greene Publishing Associates, Inc. and John Wiley & Sons, Inc., (1994 Supplement) (Ausubel); Cashion et al., U.S. Pat. No. 5,017,478; and Carr, European Patent No. 0,246,864. Examples of techniques sufficient to direct persons of skill through in vitro amplification methods are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3: 81-94; (Kwoh et al. (1989) Proc. Natl. Acad. Sci. USA 86: 1173; Guatelli et al. (1990) Proc. Natl. Acad. Sci. USA 87, 1874; Lomell et al. (1989) J. Clin. Chem., 35: 1826; Landegren et al., (1988) Science, 241: 1077-1080; Van Brunt (1990) Biotechnology, 8: 291-294; Wu and Wallace, (1989) Gene, 4: 560; and Barringer et al. (1990) Gene, 89: 117.

Detection of the Reporter Genes.

Detection of the reporter genes of this invention is by standard methods well known to those of skill in the art. Where the reporter gene is detected through its enzymatic activity this typically involves providing the enzyme with its appropriate substrate and detecting the reaction product (e.g., light produced by luciferase). The detection may involve simply detecting presence or absence of reporter gene produce, or alternatively, detection may involve quantification of the level of expression of reporter gene products. The quantification can be absolute quantification, or alternatively, can be comparitive e.g., with respect to the expression levels of one or more “housekeeping” genes. Methods of quantifying the expression levels of particular reporter genes are well known to those of skill in the art. It will be appreciated that such detection can be performed “manually” or may be automated e.g., as in a high-throughput screening system.

High throughput assays for the presence, absence, or quantification of gene expression (e.g., via the detection of the transcribed nucleic acid (mRNA) or the detection of gene expression (protein product)) are well known to those of skill in the art. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization.

Assay Kits

In another embodiment, this invention provides kits for the practice of the methods of this invention. The kits preferably include assay cells comprising an estrogen receptor, and a promoter comprising an AP1 site which regulates expression of a first reporter gene. The cell may additionally include a cognate receptor for a nuclear transcription factor. The kits may additionally include a second cell comprising an estrogen receptor, and a promoter comprising an ERE site which regulates expression of a first reporter gene. Alternatively, the kits can include one cell type that contains both the AP-1/reporter and the ERE/reporter constructs. The cells may additionally express high levels of fos and/orjun. The kits may optionally contain any of the buffers, reagents, culture media, culture plates, reporter gene detection reagents, and so forth that are useful for the practice of the methods of this invention.

In addition, the kits may include instructional materials containing directions (i.e., protocols) for the practice of the assay methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered by way of illustration, not by way of limitation.

### Example 1

Materials and Methods

Plasmids

Coll73-LUC and Coll60-CAT have been previously described (Webb et al. (1995) Mol. Endo., 9: 443-456; Lopez et al. (1993) Mol. Cell Biol., 13: 3042-3049). Coll73-LUC consists of −73 to +63 base pairs of the collagenase promoter upstream of the Luciferase reporter gene. Coll517-CAT and Coll517mAP-1-CAT each contain −517 to +63 of the collagenase promoter (Webb et al. (1995) Mol. Endo., 9: 443-456).

Coll517mAP-1-CAT contains three point mutations in the consensus AP-1 response element (TGAGTCA mutated to GTACTCA). ColALuc contains the collagenase AP-1 response element upstream of the minimal drosophila alcohol dehydrogenase promoter (Starr et al., (1996) Genes & Dev., 10: 1271-1283). The ER expression vectors have been previously described: PHE0 (Green et al. (1986) Nature, 320: 134-139), PHEG0 (Tora et al. (1989) EMBO J. 8: 1981-1986), HE11 (Kumar et al. (1986) EMBO J., 5: 2231-2236), and HE15 (Kumar et al. (1987) Cell, 51: 941-951). PHE0 contains a point mutation (Gly400Val). PHEG0 is the wild type ER. PHE0 has reduced affinity for estrogens which allows for studies in cell culture without inadvertent activation. The protein coding regions of the ER plasmids were cloned into the multiple cloning site of the pSG5 expression vector. pRSVhGR (McEwan et al. (1993) Mol. Cell. Biol., 13: 399-407) consists of a cDNA encoding the human GR coding region inserted into an expression vector driven by the Rous sarcoma virus promoter. The PR-A (PhPR-60) and PR-B (hPR65) plasmids were derived from T47D cDNA and genomic DNA (Law et al (1987) Proc. Natl. Acad. Sci., USA, 84: 2877-2881; Wei et al. (1988) Mol. Endo., 2: 62-72) and cloned into an expression vector derived from pLEN (Kushner et al. (1990) Mol. Endocrinol. 4: 1465-1473). The human c-Jun (Turner et al. (1989) Science, 243: 1689-1694)) and rat c-Fos (Cohen et al. (1989) Genes & Dev., 3: 173-184)), have been previously described. The beta-actin-hCG construct has been previously described (Lopez et al. (1993) Mol. Cell. Biol., 13: 3042-3049). The pJ3-LacZ plasmid is pBR322-based and contains an SV40 promoter which activates LacZ.

Cells

All cells were maintained in DME medium without phenol red. The medium was supplemented by serum (Sigma) which was tested for low estrogenic activity prior to use. Charcoal- and heat- (55° C.×½ hr.) treated serum was used in the GT1-1, and in all PR experiments. In these experiments cells were treated with media containing charcoal-treated serum the night prior to transfection.

Transfection

Cells were transfected by electroporation as previously described (Webb et al. (1995) Mol. Endo., 9: 443-456). Briefly, 1-2 million cells from just confluent plates were used for each cuvette. Cells were electroporated at 0.24 kV in electroporation buffer. The electroporated cells were resuspended in medium which was then divided into six well plates. The efficiency of transfection was monitored by co-transfection with either a (hCG reporter driven by an actin promoter (Lopez et al. (1993) Mol. Cell. Biol., 13: 3042-3049) or by co-transfection with pJ3LACZ. CAT or luciferase activity was then corrected by dividing by hCG levels or β-galactosidase activity. Five micrograms of collagenase reporter plasmid, and 1 μg of GR expression vector were used in all experiments unless otherwise indicated.

Cell Treatments

Cells were treated either immediately or up to 6 hours after transfection. They were then harvested at approximately 40 hrs. after plating. Dexamethasone, estradiol, and R5020 were all used at 10⁻⁷ M. Tamoxifen was used at 5×10⁻⁶ M. PMA (Sigma) was suspended in DMSO and cells were treated at 10⁻⁷ M; TNF-α (R&D Systems, Minneapolis, Minn.) was resuspended in 0.1% BSA and cells were treated at 10 ng/ml.

CAT, Luciferase, hCG, and β-Galactosidase Assays

CAT, luciferase, and hCG assays were performed as described (Webb et al. (1995) Mol. Endo., 9: 443-456; Lopez et al. (1993) Mol. Cell. Biol., 13: 3042-3049). A commercial luminescent assay (Tropix; Bedford, Mass.) was used for β-galactosidase measurements.

Data Analysis.

In most figures data has been expressed relatively to permit statistical analysis of data from separate experiments. The relative number, fold induction or percent stimulation, was averaged from 2-5 experiments as indicated in the figure legends. Standard deviation was calculated for each averaged point except for the reference which was set to one (fold induction) or 100% (percent stimulation). Fold induction was calculated as the ratio of a steroid treatment to the “No Steroid” treatment point. Percent stimulation was calculated as percent of estradiol treatment. In some figures representative data are shown instead of averaged data. This permits evaluation of the effect of a co-transfected plasmid or AP-1 activator treatment on transcription in the absence of steroid treatment. In all cases the data represented has been repeated in three or more similar experiments.

Results

GR Inhibits ER Transcriptional Activation through the AP-1 Response Element.

It has been previously demonstrated that estrogens stimulate and glucocorticoids inhibit basal activity of a truncated collagenase promoter which contains the consensus AP-1 response element (Col 173) (Gaub et al. (1990) Cell, 63: 1267-1276; Webb et al. (1995) Mol. Endo., 9: 443:456; Ponta et al. (1992) Bioch. Biophys. Acta, 1129: 255-261). Since both steroids modulate transcriptional activation through the AP-1 response element, the ability of the ER and GR to influence each other's transcriptional effects at this site was investigated.

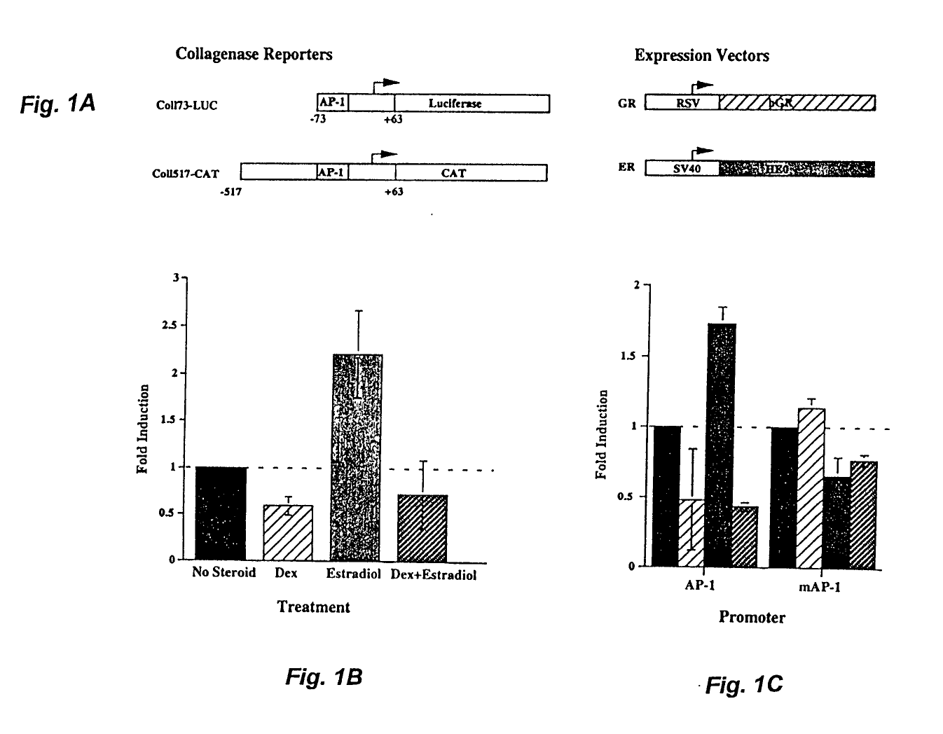

HeLa cells were transfected with ER (HE0) and the truncated collagenase promoter (Coll73-LUC) (FIG. 1A), then treated with Dex, estradiol, or Dex+ estradiol. As previously reported, Dex inhibited and estradiol stimulated transcription through this promoter. When both steroids were added, GR blocked estradiol-stimulated transcription (FIG. 1B). A similar ER-GR interaction occurs with both HE0 and HEG0, which encodes the wild type receptor.

To determine whether or not the AP-1 response element is required for the glucocorticoid/estrogen interaction, the steroid responses were evaluated at a longer form of the collagenase promoter in the presence of an intact or mutated AP-1 response element (Coll517 or Coll517mAP-1, respectively). As was the case with Coll73, Dex blocked estradiol activity through an intact AP-I response element. The steroid responses were abrogated when the promoter bearing the mutated AP-1 response element was used (FIG. 1C; (Webb et al. (1995) Mol. Endo., 9: 443-456; Ponta et al. (1992) Biochem. Biophys. Acta, 1129:255-261, and references therein)). The minor steroid effects seen in FIG. 1C are not reproducible. Steroid effects were also attenuated when HeLa cells were transfected with Coll73-CAT deleted of the AP-1 response element (Coll60-LUC; (Id.)). Dex, then, is able to block estradiol stimulation of transcriptional activation mediated by the AP-1 response element.

ER and GR Functionally Compete at the AP-1 Response Element.

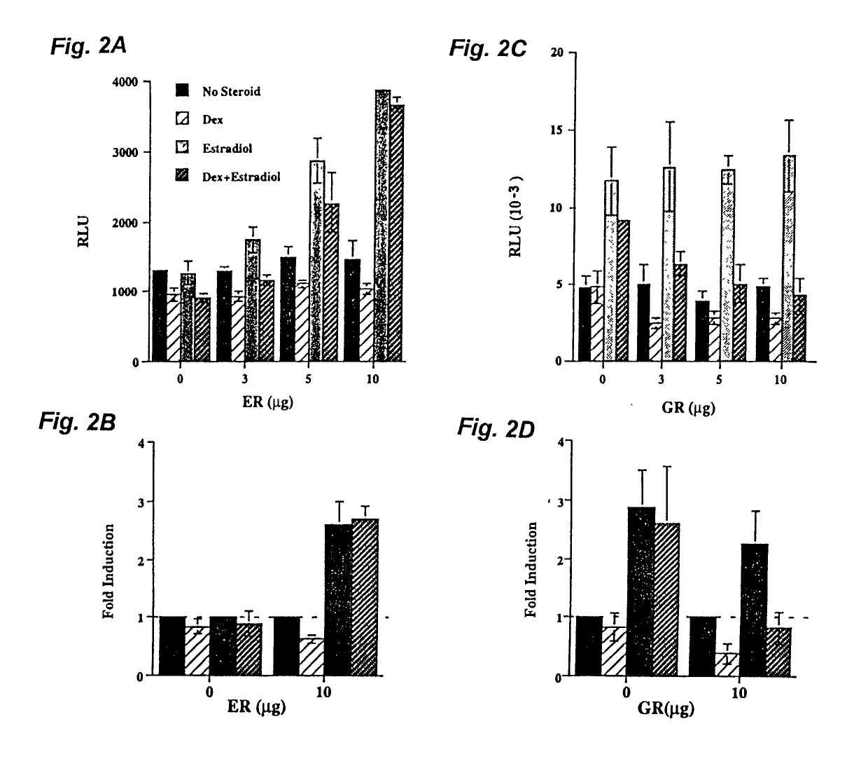

The above finding that Dex could block estradiol stimulation of transcriptional activity at the AP-1 site suggested that the ER and GR might functionally compete at this response element. In order to determine whether or not this was the case, HeLa cells were transfected with increasing amounts of ER in the presence of a constant amount of co-transfected GR (1 μg). At high levels of transfected ER, Dex was unable to inhibit the estradiol response (FIG. 2A and FIG. 2B). Then increasing amounts of GR were transfected in the presence of a constant, high level of co-transfected ER. In the presence of endogenous levels of GR, Dex was unable to inhibit estradiol stimulation. Dex inhibition was restored by co-transfecting >1 μg of GR and became more pronounced at higher levels of GR (FIG. 2C and FIG. 2D and data not shown). Taken together, these data and the data presented in FIG. 1 indicate that ER and GR transcriptional actions functionally compete through the AP-1 response element. The competitive nature of this interaction predicts that the net outcome of estrogen and glucocorticoid transcriptional activity at the AP-1 response element will depend on the ratio of ER to GR in a given cell. High levels of ER would result in stimulation and high levels of GR would result in inhibition. Intermediate levels of each would result in an intermediate effect. In some cases a given proportion of ER:GR might resulting in the cancellation of any estrogen or glucocorticoid effects at all.

Dexamethasone Inhibits Estradiol- and Tamoxifen-Mediated ER Activation through the AP-1 Response Element.

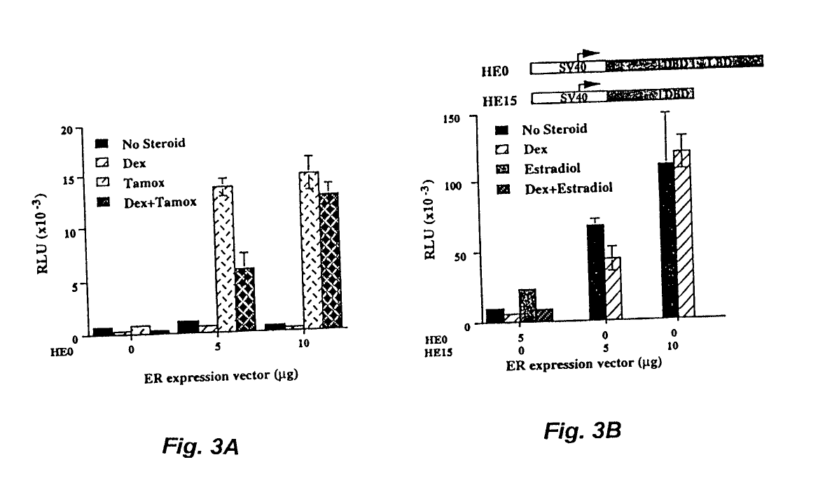

It has been proposed that ER stimulation of transcription through the AP-1 response element occurs through more than one pathway (Webb et al. (1995) Mol. Endo., 9: 443-456). The alpha pathway is characterized by tamoxifen-induced transcriptional activation and a requirement for the ER DNA binding domain. Dex inhibited tamoxifen activation (FIG. 3A). As seen with estradiol, the degree of Dex inhibition diminished in the presence of high levels of co-transfected ER (FIG. 3A). A C-terminally deleted ER (HE15) serves as a model of tamoxifen activation. It lacks the activation function in the C-terminus and activates transcription through the activation function in the N-terminal domain. Therefore, it is constitutively active at Coll73 (Id.). When cells were transfected with increasing amounts of HE15 and treated with vehicle or Dex, Dex inhibited the constitutive activity of HE 15 (FIG. 3B). Again, the interaction was functionally competitive; over-expression of HE15 (FIG. 3B) overcame Dex mediated inhibition.

Dexamethasone Inhibits the Beta Pathway Characterized by Estradiol Activation and the Lack of a Requirement for the ER DNA Binding Domain.

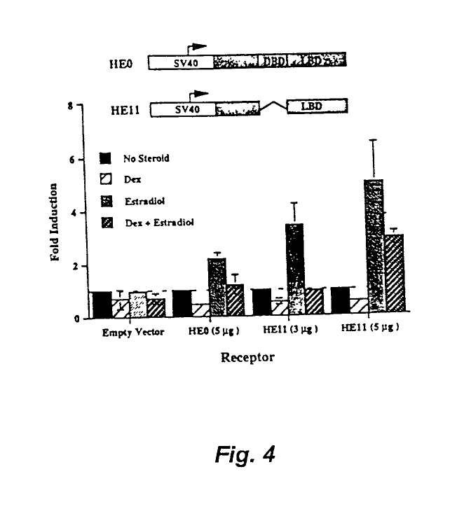

To determine whether or not Dex could inhibit estradiol-liganded HE11 (which lacks the DNA binding domain), cells were treated with estradiol, Dex, or Dex+estradiol, as above. Dex inhibited estradiol activation through HE11 (FIG. 4). Since Dex inhibited tamoxifen stimulation, the constitutive activity of the ER deleted of its C-terminal domain, and estradiol-activated ER deleted of its DNA binding domain (FIGS. 3 and 4), it is inferred that glucocorticoids can inhibit both alpha and beta pathways of ER stimulation.

c-Jun and c-Fos Differentially Alter Estradiol and Dex Effects

It has been demonstrated that individual members of the AP-1 family differentially change the pattern of steroid receptor activation at a hormone response element. For example, increasing amounts of c-Jun and c-Fos progressively attenuate ER activation at an estrogen response element (ERE) in MCF-7 cells whereas transfected JunD does not (Doucas et al. (1991) EMBO J, 10: 2237-2245). In addition, the ratio of Jun:Fos in a given cell will change the steroid response to Dex at the AP-1 site (Teurich et al., (1995) Chem. Senses, 20: 251-255) and the proliferin composite (GRE/AP-1) response element (Diamond et al. (1990) Science, 249: 1266-1272).

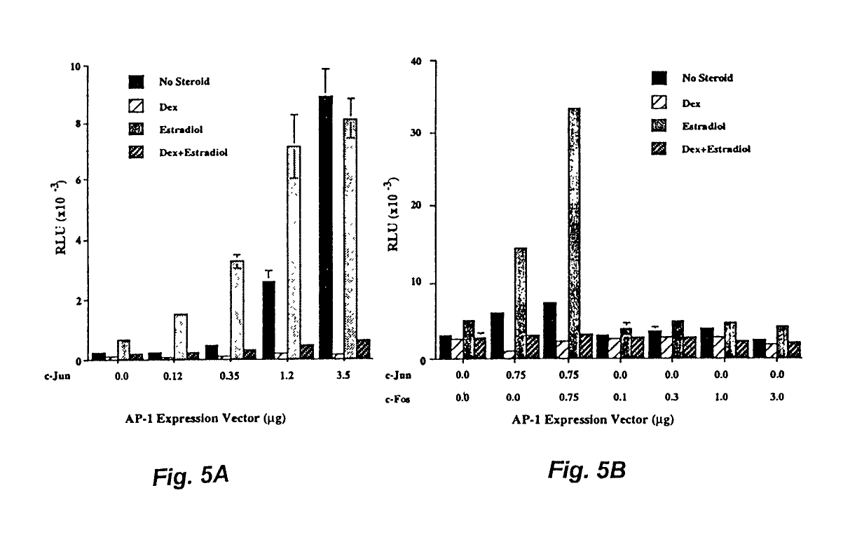

Steroid responses were evaluated in the presence of increasing amounts of transfected c-Jun or c-Fos expression vectors. As previously demonstrated, c-Jun increased estradiol transcriptional activation at CoII73 (FIG. 5A). At levels of co-transfected c-Jun which resulted in slightly increased AP-1 activated transcription, estradiol stimulation was potentiated. At levels of co-transfected c-Jun which resulted in marked stimulation of AP-1 activated transcription, further estradiol stimulation of AP-1 activation was no longer present. Dex treatment alone restricted transcriptional activity to low levels at all amounts of transfected c-Jun. In the presence of both Dex and estradiol, the levels of transcription were close to those seen when cells were treated with Dex alone. Co-transfected c-Fos potentiated c-Jun stimulation of estradiol-mediated transcriptional activation (FIG. 5B; Webb et al. (1995) Mol. Endo. 9: 443-456). In distinction to transfection with c-Jun alone, transfection with c-Fos alone failed to alter steroid responses. Co-transfection of Jun B and D (0.1-3.0 μg) had minimal effects on the pattern of steroid responses. Therefore, individual AP-1 family members appear to have different effects on the profile of steroid responses at the AP-1 site.

Activators of c-Jun Differentially Alter Estradiol and Dex Patterns of Response at the AP-1 Response Element.

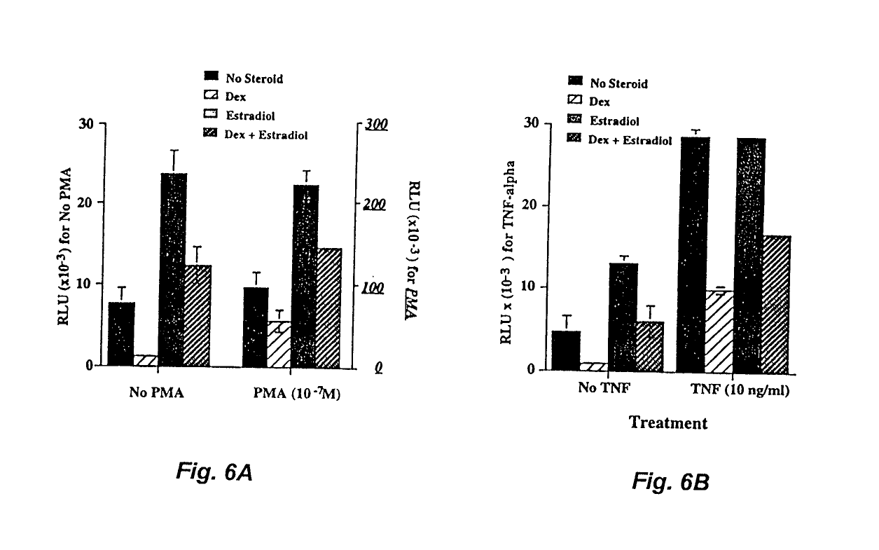

The phorbol ester PMA, and the cytokine tumor necrosis factor-alpha (TNF-α both activate c-Jun. However, they do so through different pathways which ultimately target different c-Jun phosphorylation sites (Boyle et al. (1991) Cell, 64: 573-584, Westwidk et al. (1994) J. Biol. Chem., 269: 26396-26401). To determine whether or not glucocorticoid and/or estrogen effects at the AP-1 response element would be altered in the presence of these activators, HeLa cells were treated with estradiol and/or Dex in the presence or absence of either PMA (10⁻⁷ M) or TNF-α (10 ng/ml). These doses resulted in maximal AP-1 activation for each agent (data not shown). PMA treatment in the absence of steroids resulted in a ten fold stimulation of transcriptional activity (FIG. 6A, note difference in the scale of the No PMA and PMA axes). The pattern of steroid effects was maintained in the presence of PMA (FIG. 6A). In distinction, estradiol stimulation was no longer apparent in the presence of TNF-α although Dex inhibition was maintained (FIG. 6B). The loss of estradiol stimulation was not a result of altering the functional activity of ER. Cells simultaneously transfected with both Coll73-LUC and ERE-Coll60-CAT failed to show diminished activity of ER at an ERE (data not shown). Therefore, while both of these agents activate c-Jun, they each have different effects on estradiol responses at the AP-I response element.

The GR Inhibits ER Stimulation in a Hypothalamic Cell Line.

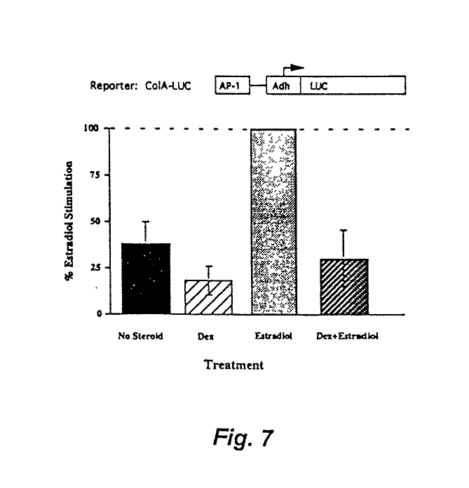

To determine whether or not the ER/GR/AP-1 response element interaction was restricted to HeLa cells, the initial experiments were repeated in a hypothalamic cell line. GT1-1 cells were derived from a transgenic mouse whose GNRH neurons were targeted for transformation by the SV40 T antigen (Mellon et al. (1990) Neuron 5: 1-10). They express neuronal but not glial markers (Id.), GNRH (Id.), and the glucocorticoid receptor (Chandran et al., (1994) Endocrinol., 134: 1467-1474). GT1-1 cells were transfected with the reporter plasmid CoIALuc (Starr et al. (1996) Genes & Dev., 10: 1271-1283), ER, and GR. In the absence of co-transfected c-Jun, estradiol stimulation or Dex inhibition of either basal or estradiol stimulated transcription was not observed. In the presence of transfected c-Jun, the pattern of steroid responses was similar to that seen in HeLa cells (FIG. 1A): estradiol stimulated, and Dex inhibited both basal and estradiol stimulated transcription (FIG. 7). Like HeLa cells (FIG. 1B), GT1-1 cells transfected with a collagenase reporter bearing a mutated AP-1 response element (Coll517mAP-1) failed to show steroid responses when compared to Coll517. These data suggest that in the appropriate state of c-Jun expression, ER and GR may competitively interact to modulate expression of genes activated through the AP-I response element in neurons.

The Progesterone Receptor (PR) Interacts with ER at the AP-1 Site.

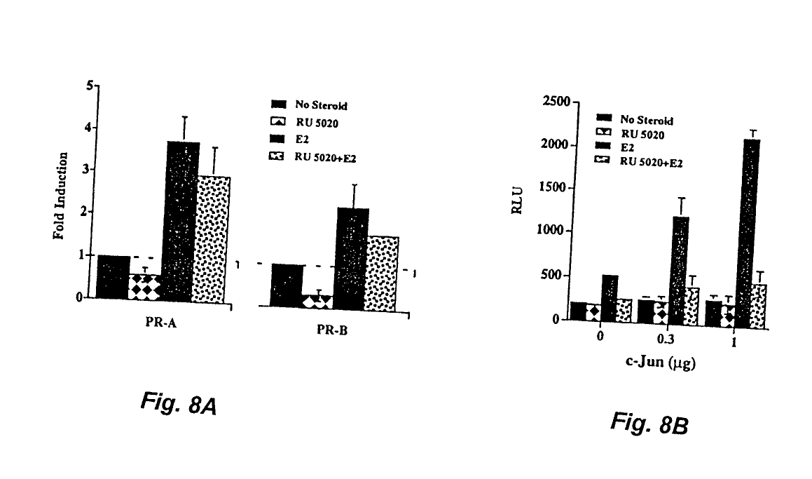

Like the glucocorticoids, progestins oppose estrogen actions. Since it has been demonstrated that the PR inhibits PMA activated transcription through the AP-1 response element (Bamberger et al. (1996) Proc. Natl. Acad. Sci., USA, 93: 6169-6174), it was determined whether or not the PR could also interact with the ER at the AP-1 response element. In HeLa cells, the progesterone agonist R5020 inhibited the basal activity of an AP-1 site through both PR-A and PR-B (FIG. 8A). As before, estradiol treatment stimulated transcription. Treatment with both steroids resulted in a loss of RU5020 inhibition. PR-A behavior was then evaluated in a different cell line. In the presence of transfected PR-A, R5020 inhibited the estradiol response in CV-1 cells monkey kidney cells which lack endogenous GR (FIG. 8B). Further, co-transfection of increasing amounts of c-Jun resulted in a pattern similar to that seen in HeLa cells (compare FIG. 5A to 8B): transfecting increased amounts of c-Jun lead to increased estradiol stimulation while RU5020 inhibition of estradiol was maintained at low levels. As in the case of the GR, PR-A responses were not seen in the presence of the collagenase reporter bearing the mutated AP-1 site (Coll517 vs. Coll517mAP-1). These data indicate that, like ER and GR, ER and PR influence each other's transcriptional activation properties at the AP-1 response element.

Discussion

These experiments demonstrate that the ER functionally interacts with the GR and PR at the consensus AP-I response element. The ER/GR interaction is functionally competitive. Dex inhibits more than one ER ligand and receptor form, and Dex inhibits ER activation potentiated by co-transfected c-Jun. These experiments further demonstrated that neither ER/GR nor ER/PR-A interactions were limited to HeLa cells.

The data presented here support the hypothesis that opposing effects of estrogens and glucocorticoids or progestins can be mediated at the level of transcription. It has been previously reported that the ER, GR and PR compete for unidentified factors involved in transcriptional regulation at hormone response elements (Meyer et al. (1989) Cell, 57: 433-442). Here it is shown that estrogen and glucocorticoid or progesterone receptors influence each other's activity at an element through which they individually regulate transcription: the AP-1 site. This does not preclude that steroid interactions occur through other mechanisms, some of which may include other nuclear transcription factors.

The potential implications of these results are several fold. First, while a cell may be capable of mounting an estrogen or glucocorticoid response at the AP-1 response element, whether or not the response will actually occur will depend on the relative levels of each receptor. Estrogen stimulation of AP-1 regulated genes may be blunted in the presence of glucocorticoids. Conversely, glucocorticoid inhibition could be overcome by estrogen activation. Second, the steroid response will be modulated by the levels and composition of the AP-1 protein complex in the cell. Transfected c-Jun and c-Fos differentially altered the estrogen and glucocorticoid pattern of transcription. Lastly, the steroid responses will also be modified by the activation state of the cell. Certain activators of AP-1 may modulate a steroid response, e.g. TNF-α modulation of estrogen stimulation, while others may not.

There are several candidate genes for which such an ER/GR/ or ER/PR/AP-1 response element interaction might be important. In the uterus, estradiol treatment increases the level of IGF-1 mRNA and the increase is attenuated by prior administration of Dex (Sahlin (1995) J. Steroid Biochem. Molec. Biol., 55: 9-15). Our data from HeLa cells, a cell line originally derived from a uterine cervical adenocarcinoma, suggests that genes expressed in the uterus have the cellular machinery to integrate ER and GR or PR responses through the AP-1 response element. The ER/PR interaction would be particularly important to pursue in uterine tissues given the number of physiologic estrogen/progestin interactions in that organ. For example, the high estrogen levels of the menstrual follicular phase are associated with proliferation of the endometrial epithelium. The transition from the proliferative to the secretory phase is associated with increased levels of progesterone. It is possible that genes associated with this transition are jointly regulated by estrogen and progesterone at an AP-1 site.

In the nervous system, estrogens and glucocorticoids regulate the synthesis of numerous neuropeptides including VP, POMC and GNRH (Brot et al. (1993) Peptides, 14: 933-940; Albeck et al. (1994) Mol. Brain. Res., 26: 129-134; Wilcox et al (1985) Endocrinol., 117: 2329-2396). Since these experiment show that the ER/GR/AP-1 response element interaction is present in a hypothalamic cell line, it is possible that neurons which express these genes could have the cellular machinery to integrate estrogen and glucocorticoid or progestin effects at AP-1 sites. In particular, GT1 cells synthesize GnRH. Evidence exists that they contain functional endogenous ER. Further, GT1 cells contain endogenous GR, which apparently functions to down-regulate GnRH transcription in GT1 cell lines in response to Dex (Chandran et al. (1994) Endocrinol, 134: 1467-1474). The present experiments indicate that GnRH, which contains an AP-1 response element in its promoter (Bruder et al. (1992) Endocrinol, 131: 2552-2558), could be regulated by estrogens and glucocorticoids in this manner.

The data presented here demonstrate that the AP-1 response element integrates the transcriptional properties of the ER with three other members of the nuclear receptor transcription factor family, the GR and PR-A and PR-B. Multiple receptors in this family have been shown to act at an AP-1 site (Lopez et al. (1993) Molec. Cell. Biol., 13: 3042-3049; Ozono et al. (1990) J. Biol. Chem., 265: 12881-21888; Schule et al. 91990) Cell, 61: 497-504; Schule et al. (1992) Proc. Natl. Acad. Sci., USA, 88: 6092-6096; Kallio et al. (1995) Mol. Endo., 9: 1017-1028). Therefore, it is expected that the AP-1 response element will integrate the effect of the ER with other members of the family, as well as to integrate the effects of other superfamily members with each other. Such integration might occur at Jun/Jun, Jun/Fos AP-1 complexes or through shared co-activators. For example, the CREB-Binding Protein (CBP) is a co-activator for AP-1 (Arias et al. (1994) Nature, 370: 226-229). In turn, CBP has been shown to interact with several members of the steroid receptor superfamily as well as with members of the steroid receptor co-activator (SRC) family (Kamei et al., (1996) Cell, 85:; 403-414). Therefore, the functional interaction of the steroid receptors described at the AP-1 site could be mediated not only through AP-1 protein complexes but also through a number of co-activator proteins involved in transducing steroid receptor signals to the basal transcriptional machinery.

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

What is claimed is:

1. A method of screening a nuclear transcription factor ligand for the ability to modulate estrogen activation at an AP-1 site, said method comprising the steps of:
a) providing a first cell containing an estrogen receptor, a receptor for said nuclear transcription factor ligand, and a promoter comprising an AP-1 site which regulates expression of a first reporter gene;
b) contacting said first cell with said transcription factor ligand and with a compound having AP-1 mediated estrogenic activity; and
c) detecting expression of said first reporter gene.

2. The method of claim 1, further comprising the steps of:
d) providing a second cell containing an estrogen receptor, a receptor for said nuclear transcription factor ligand, and a promoter comprising an estrogen response element (ERE) that regulates expression of a second reporter gene;
e) contacting said second cell with said transcription factor ligand and with said compound having AP-1 mediated estrogenic activity; and
f) detecting expression of said second reporter gene.

3. The method of claim 2, wherein said first cell and said second cell are the same cell.

4. The method of claim 1, further comprising the steps of:
d) providing a second cell containing a cognate receptor of said transcription factor ligand, and a promoter comprising a response element for said cognate receptor that regulates expression of a second reporter gene;
e) contacting said second cell with said transcription factor ligand and with said compound having AP-1 mediated estrogenic activity; and
f) detecting expression of said second reporter gene.

5. The method of claim 4, wherein said first cell and said second cell are the same cell.

6. The method of claim 1, wherein said nuclear transcription factor ligand is selected from the group consisting of a glucocorticoid, a progestin, vitamin D, retinoic acid, a an androgen, a mineralcorticoid, and a prostangladin.

7. The method of claim 1, wherein said cognate receptor is selected from the group consisting of an estrogen receptor, a glucocorticoid receptor, a progestin PR-A receptor, and progestin PR-B receptor, androgen receptor, a mineralcorticoid receptor, and a prostaglandin receptor.

8. The method of claim 1, wherein said cell expresses said estrogen receptor from a heterologous DNA.

9. The method of claim 1, wherein said cell expresses said cognate receptor from a heterologous DNA.

10. The method of claim 1, wherein said cell expresses an AP-1 protein from a heterologous DNA.

11. The method of claim 10, wherein said AP-1 protein is c-jun.

12. The method of claim 1, wherein said nuclear transcription factor is a progestin; and said cognate receptor is a progestin receptor.

13. The method of claim 1, wherein said nuclear transcription factor is a glucocorticoid and said cognate receptor is a GR receptor.

14. A method of screening an agent for the ability to alter modulation of estrogen activation at an AP-1 site by a nuclear transcription factor ligand, said method comprising the steps of:
a) providing a first cell containing an estrogen receptor, a receptor for said nuclear transcription factor ligand, and a promoter comprising an AP-1 site which regulates expression of a first reporter gene;
b) contacting said first cell with said transcription factor ligand, with a compound having AP-1 mediated estrogenic activity, and with said agent; and
c) detecting expression of said first reporter gene.

15. The method of claim 14, further comprising the steps of:
d) providing a second cell containing an estrogen receptor, a receptor for said nuclear transcription factor ligand, and a promoter comprising an estrogen response element (ERE) that regulates expression of a second reporter gene;
e) contacting said second cell with said transcription factor ligand and with said compound having AP-1 mediated estrogenic activity; and
f) detecting expression of said second reporter gene.

16. The method of claim 15, wherein said first cell and said second cell are the same cell.

17. The method of claim 14, wherein said nuclear transcription factor is selected from the group consisting of a glucocorticoid, a progestin, vitamin D, retinoic acid, an androgen, a mineralcorticoid, a prostaglandin.

18. The method of claim 14, wherein said cognate receptor is selected from the group consisting of an estrogen receptor, a glucocorticoid receptor, a progestin PR-A receptor, progestin PR-B receptor, an androgen receptor, a mineralcorticoid receptor, and a prostaglandin receptor.

19. The method of claim 14, wherein said cell expresses said estrogen receptor from a heterologous DNA.

20. The method of claim 14, wherein said cell expresses said cognate receptor from a heterologous DNA.

21. The method of claim 14, wherein said cell expresses an AP-1 protein from a heterologous DNA.

22. The method of claim 21, wherein said AP-1 protein is c-jun.

23. The method of claim 21, wherein said nuclear transcription factor is a progestin; and said cognate receptor is a progestin receptor.

24. The method of claim 21, wherein said nuclear transcription factor is a glucocorticoid and said cognate receptor is a GR receptor.

25. A method of screening an orphan receptor for the ability to modulate estrogen activation at an AP-1 site, said method comprising the steps of:
a) providing a first cell containing an estrogen receptor, and orphan receptor, and a promoter comprising an AP-1 site that regulates expression of a first reporter gene;
b) contacting said first cell with a compound having AP-1 mediated estrogenic activity; and
c) detecting expression of said first reporter gene.

26. A kit for screening a nuclear transcription factor ligand for the ability to modulate estrogen activation at an AP-1 site, said kit comprising:
a first cell containing an estrogen receptor, a receptor for said nuclear transcription factor ligand, and a promoter comprising an AP-1 site which regulates expression of a first reporter gene; and
instructional materials containing protocols for the practice of the assay methods of claim 1.

27. The kit of claim 26, wherein said instructional materials further contain protocols for the practice of the assay method of claim 2.

28. The kit of claim 26, wherein said instructional materials further contain protocols for the practice of the assay method of claim 4.
